(19) United States
(12) Patent Application Publication
(10) Pub. No.: US 2012/0168331 A1
(43) Pub. Date: Jul. 5, 2012

(54) Voice template protector for administering medicine

(75) Inventor: Uri Tagger, Even Yehuda (IL)

(73) Assignee: SAFECODE DRUG TECHNOLOGIES CORP., Newark, NJ (US)

(21) Appl. No.: 12/982,158

(22) Filed: Dec. 30, 2010

(51) Int. Cl. A61B 19/02

(52) U.S. Cl. 206/364; 206/438

(57) ABSTRACT

This invention relates generally to systems for imporving the safety in medication use. Specifically, the invention relates to the use of matching drug name voice templates as a threshold mechanism for unlocking a medication container.

### FIELD OF INVENTION

This invention is generally directed to systems for imporving the safety in medication use. Specifically, the invention is directed to the use of matching drug name voice templates as a threshold mechanism for unlocking a medication container.

### BACKGROUND OF THE INVENTION

In 2005 alone, causes of prescription error that involve some aspect of communication (e.g., verbal orders, illegible handwriting abbreviations, look-alike or sound-alike names) comprised 37% of all error causes, considered by some to be the top error cause. In some cases, the problem of sound alike drugs is further compounded when the dosesand packages for these drugs are similar. These errors can occur at any point in the medication use system. In certain circumstances, such as in ER triage and other pressure situation, the transcription errors may be compounded further and lead in severe cases to fatalities. By 2006, the number of Look-alike/Sound alike (LASA) drug pairs (e.g. Mellaril/Elavil, Paxil/Taxol, Prilosec/Prozac, and Celebrex/Celexa) was reported to be over 3000 and the number of transcription errors resulting in fatalities due to LASA was reported to be over 350.

The LASA problem may be exacerbated when drugs are imported from overseas. Americans traveling outside the United States or purchasing drugs over the internet return with, or receive medications purchased abroad. They may think these medications are identical to US products with brand names that are the same or similar, but often this is not the case. Examples include Dianben (metformin) and Diovan (valsartan) in Spain, Avanza (mirtazapine) and Avandia (rosiglitazone) in Australia, and Trip (nortriptyline) and Triz (cetirizine) in India.

By 2010, of the more than 5 million wrong-drug errors that occur each year, many were reported to be caused by similar-sounding mixed-up names. These prescription errors of, for example confusing the migraine drug Topamax with the blood pressure drug Toprol-XL, or the antihistamine Zyrtec with the antipsychotic Zyprexa, continue to plague healthcare professionals, causing substantial morbidity and mortality.

There is a need for an additional layer of protection during the medication use system to address these issues.

### SUMMARY OF THE INVENTION

In one embodiment, the invention provides a This invention is generally directed to systems for imporving the safety in medication use. Specifically, the invention is directed to the use of matching drug name voice templates as a threshold mechanism for unlocking a medication container.

In another embodiment, the invention provides a system for providing patient-specific drugs, comprising: a drug container, wherein the drug container comprises a locking means; a patient identifying barcode; a drug name identifier; and a computer interface; and a computer, wherein the drug container is connected to the computer and the locking means opens in response to a signal from the computer.

In one embodiment, the invention provides a system for providing patient-specific drugs, comprising: a drug container, wherein the drug container comprises a locking means; a patient identifying barcode; a drug name identifier, wherein the drug name identifier comprises: a computer readable medium containing a stored word template or templates identifying the drug trade name, the compound name or both; and a processing unit capable of sending the stored template or templates to a computer; and a computer interface; and a computer, wherein the drug container is connected to the computer through the computer interface and the locking means opens in response to a signal from the computer following a positive comparison between the stored template or templates and a voice sample identifying the drug provided by a reference person, such as the drug prescriber or the drug administrator.

### BRIEF DESCRIPTION OF THE DRAWINGS

The invention will be better understood from a reading of the following detailed description taken in conjunction with the drawings in which like reference designators are used to designate like elements, and in which:

FIG. 1 shows a schematic representation of an embodiment of the system; and

FIG. 2 shows the function of the pattern matching block of the isolated-word speech recognition system used to determine the similarity between the input word pattern 700 and the stored patterns of the drug brand name, generic drug name or their combination.

### DETAILED DESCRIPTION OF THE INVENTION

This invention relates in one embodiment to systems for imporving the safety in medication use. In another embodiment, the invention relates to the use of matching drug name voice templates as a threshold mechanism for unlocking a medication container.

In one embodiment, provided herein is a system for providing patient-specific drugs, comprising: a drug container, wherein the drug container comprises a locking means; a patient identifying barcode; a drug name identifier; and a computer interface; and a computer, wherein the drug container is connected to the computer and the locking means opens in response to a signal from the computer.

Placement and use of the patient identifying bar-code as described hereinabove, is provided in U.S. Pat. No. 7,347,841 incorporated herein by reference in its entirety.

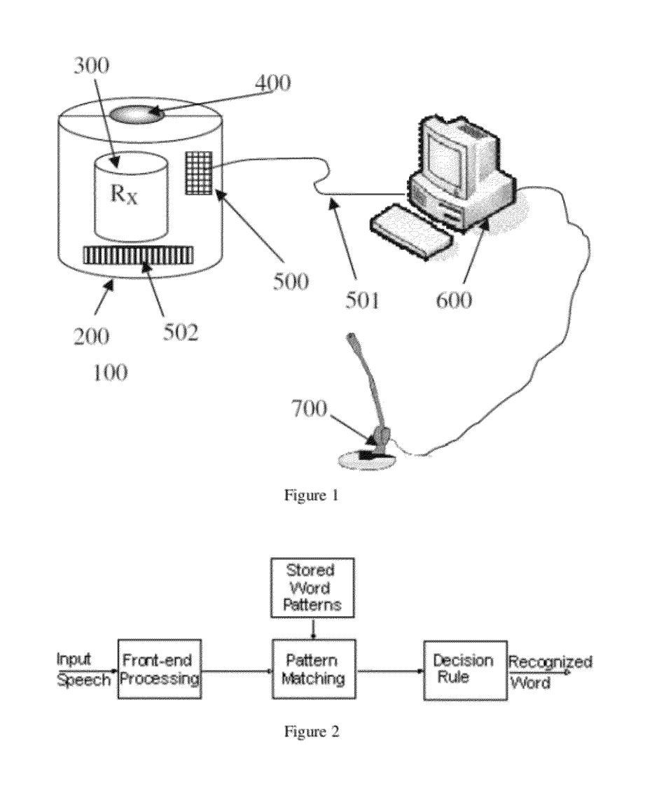

Reference is now made to FIG. 1, showing the system 100, comprising a lockable drug container 200, wherein the drug to be prescribed 300 is located. The drug container 200 is locked with a locking means 400, capable of opening the container upon receiving a signal from a computer 600 located in the vicinity of the container 200. Drug 300 identifying means such as a patient identifying bar-code 502 in one embodiment, is displayed on container 200 and is capable of being scanned by an appropraiet reader to verify the patient receiving the drug 300 is the correct patient. Drug name identifier 501 is affixed in the container or on the container and is in communication with a processing unit 500 and a computer interface 501. The computer interface may be a hard wire connected via USB cable or any other appropriate communication means between the container 200 and the computer 600. This may be a wireless communication device as well. Upon receiving a signal from the computer 600, the lock 400 on container 200 may open and allow drug 300 to be administered to a patient in need thereof. In one embodiment, drug container 200 is a pill box, a syringe, infusion bag and valve, a vial, an ampule or a combination thereof.

In another embodiment, provided herein is a system for providing patient-specific drugs, comprising: a drug container 200, wherein the drug container comprises a locking means 400; a patient identifying barcode 502; a drug name identifier 500, wherein the drug name identifier 500 comprises: a computer readable medium containing a stored word template or templates identifying the drug 300 trade name, the compound name or both; and a processing unit capable of sending the stored template or templates to a computer 600; and a computer interface 501; and a computer 600, wherein the drug container 200, such as a drug dispensing machine is connected to the computer through the computer interface 501 and the locking means 400 opens in response to a signal from the computer 600 following a positive comparison between the stored template or templates and a voice sample identifying the drug provided 700 by a reference person, such as the drug prescriber or the drug administrator.

In another embodiment, the term “voice templates” refers to a software program and associated data structures for enabling a user easily to build and maintain voice responses associated with a drug brand name, generic drug name or their combination. Such templates may be decomposed “voice elements” by the user into of various types, the characteristics and behavior of which will be determined by their specific type, as designated by the user, and the characteristic of the voice response record with which they are associated and compared to.

The computer 600 includes in one embodiment typical hardware such as a motherboard, central processing unit, random access memory, a hard disk, network card, power supply and visual display unit. In another embodiment, computer 600 also includes a microphone 700 for receiving voice commands and recording voice templates. In addition to the hardware, the hard disk of each computer 600 is loaded with an operating system and browser software which interact with the systems described herein to allow computer 600 to connect to the Internet and access a centralized depository of stored voice templates of drug brand name, generic drug name or their combination. The computer 600 includes in one embodiment, driver software for an audio recording program, such as voice recognition software for interacting with the microphone to record the voice templates. As previously discussed, the communication system between the drug identifying means 500 is in the form of a public access packet-switched network, such as the Internet. Computer 600 is able in another embodiment to exchange data (e.g. XML code and packetised voice messages) with the voice template stored on the drug identifying means 500 using a packet-switched network protocol, such as the TCP/IP protocol. In an alternative embodiment (not shown), the communication system may additionally comprise a third generation (“3G”) or GPRS enabled mobile telephone network connected to the packet-switched network which can be utilised to access the voice template stored on the drug identifying means 500. In such an embodiment, computer 600 would have wireless capabilities for transmitting the voice input. The wireless computing devices may include, for example, mobile phones, personal computers having wireless cards and any other mobile communication device which facilitates voice recordation functionality. In another embodiment, the present invention may employ an 802.11 based wireless network or some other personal virtual network.

In one embodiment, provided herein is a system for providing patient-specific drugs, comprising: a drug container 200, wherein the drug container comprises a locking means 400; a patient identifying barcode 502; a drug name identifier 500, wherein the drug name identifier 500 comprises: a computer readable medium containing a stored word template or templates identifying the drug 300 trade name, the compound name or both; and a processing unit capable of sending the stored template or templates to a computer 600; and a computer interface 501; and a computer 600, wherein the drug container 200 is connected to the computer through the computer interface 501 and the locking means 400 opens in response to a signal from the computer 600 following a positive comparison between the stored template or templates and a voice sample identifying the drug provided 700 by a reference person, wherein using a predetermined algorithm (see e.g. FIG. 2), the computer 600 digitizes the reference voice and compares the pattern of the stored drug name, brand name, compound name or their combination against at least one reference voice encoded to enable the comparison.

In one embodiment, a voice template may be created by using parameters such as cepstral coefficients in an acoustic vector sequence obtained by performing acoustic analysis on speech signals output from a speaker. In another embodiment, the algorithm used to provide the positive comparison between the digitizes the stored drug name, brand name, compound name or their combination and at least one reference voice encoded to enable the comparison, is dynamic time warping (DTW). DTW refers in one embodiment to the technique used to account for speaker variations and concerns the operation of compressing or stretching the temporal pattern of speech signals to take speaker variations into account. In certain embodiments, pattern or Template of each word identifying the drug brand name, generic drug name or their combination is stored as a time sequence of features (frames), derived using speech analysis techniques such as source filter model in one embodiment, or Perceptual-linear-predictive technique in another embodiment, or relative spectral PLP technique in yet another embodiment. Recognition is performed in one embodiment by comparing the acoustic pattern of the drug brand name, generic drug name or their combination to be recognized with the stored patterns and choosing the word which it matches best as the recognized word. In another embodiment, an ‘average’ set of patterns is previously stored in the drug identifier 500 and no training is required of a speaker. The function of the pattern matching block of the isolated-word speech recognition system in the figure below is to determine the similarity between the input word pattern and the stored word patterns.

In one embodiment, the locking means 400 used with the drug container 200, will open upon the determination that there is a match between the stored template of the drug brand name, generic drug name or their combination and the reference voice provided by the drug prescriber, the drug administrator, the patient or any other similarly situated healthcare professional.

In one embodiment, provided herein is a method for providing a specific drug to a specific patient, comprising the step of obtaining a computer readable from a prescribing healthcare professional, the computer readable media having a word template of a drug brand name, generic drug name or their combination stored thereon, obtaining a drug container 200, wherein the drug container comprises a locking means 400; a patient identifying barcode 502; a drug name identifier 500, wherein the drug name identifier 500 comprises: a computer readable medium containing a stored word template or templates identifying the drug 300 trade name, the compound name or both; and a processing unit capable of sending the stored template or templates to a computer 600; and a computer interface 501; and a computer 600, wherein the drug container 200 is connected to the computer through the computer interface 501 and the locking means 400 opens in response to a signal from the computer 600; comparing the word template stored on the drug identifier against the drug brand name, generic drug name or their combination stored on the computer readable media obtained from the prescribing phycian and following a positive comparison between the stored template or templates and the readable media templae, opening the lock to the drug container 200 and retreiving the drug 300.

In one embodiment, the templates of the drug brand name, generic drug name or their combination, are derived by first obtaining a large number of sample patterns from a cross-section of healthcare providers in a given facility where the drugs are to be administered; of different sex, age-group and dialect, and then clustering these to form a representative pattern for each of the drug brand name, generic drug name or their combination. A representative pattern can be created by averaging all the patterns in a cluster. A dynamic time-warping algorithm is employed in another embodiment to compute a time-alignment function which takes into account the different time-scales. In another embodiment, a representative pattern is selected from the middle of each cluster. Because of the great variability in speech, it is generally impossible to represent each word cluster with a single pattern. Thus, in one embodiment each cluster is sub-divided into sub-clusters and a number of tokens for each drug brand name, generic drug name or their combination, i.e. up to twelve, is stored in the drug identifying means. In one embodiment, all tokens of each stored drug brand name, generic drug name or their combination are matched against the drug brand name, generic drug name or their combination obtained from the prescribing healthcare provider stored at the time of prescription on the computer readable media.

In one embodiment, the computer used in the systems and methods provided herein comprises a CPU; a speech recognition means for recognizing an inputted speech, and storage means for storing a plurality of templates of drug brand name, generic drug name or their combination, wherein: said CPU displays a set of templates of drug brand name, generic drug name or their combination that are related to a first inputted speech recognized by said speech recognition means; said speech recognition means recognizes a second inputted speech while the set of templates of drug brand name, generic drug name or their combination are being displayed, by comparing with acoustic analysis of the displayed templates and selecting a template similar to said second inputted speech; and said CPU decodes said recognized result and sends a signal to the locking means 400 to open a drug container 200.

In one embodiment, the computer readable medium, is a computer readble recording medium having stored thereon an inputted drug name, a generic drug name, or a combination thereof. In another embodiment, the inputted drug brand name, generic drug name or their combination is recorded by a prescribing physician.

Having described preferred embodiments of the invention with reference to the accompanying examples, it is to be understood that the invention is not limited to the precise embodiments, and that various changes and modifications may be effected therein by those skilled in the art without departing from the scope or spirit of the invention as defined in the appended claims.

What is claimed is:

1. A system for providing patient specific drugs, comprising:
a. A drug container, wherein the drug container comprises
i. a locking means;
ii. a patient identifying barcode;
iii. a drug name identifier; and
iv. a computer interface; and
b. a computer, wherein the drug container is connected to the computer and the locking means opens in response to a signal from the computer.

2. The system of claim 1, wherein the drug name identifier comprises:
a. A computer readable medium containing a stored word template identifying the drug brand name, generic drug name or their combination; and
b. A processing unit capable of sending the template to the computer

3. The system of claim 2, wherein using a predetermined algorithm, the computer compares the pattern of the recorded voice against at least one reference voice.

4. The system of claim 3, wherein the reference voice is provided by a person prescribing the drug, the drug administering person or both.

5. The system of claim 4, wherein the algorithm uses dynamic time warping (DTW).

6. The system of any one of claims 1-5, wherein the locking means will open upon the determination that there is a match between the stored word template and the reference voice.

7. The system of any one of claims 1-5, wherein the drug container is a pill box, a syringe, infusion bag and valve, a vial, an ampule or a combination thereof.
